(19) United States
(12) Patent Application Publication
(10) Pub. No.: US 2014/0179981 A1
(43) Pub. Date: Jun. 26, 2014

(54) METHOD AND SYSTEM FOR POSITIONING A TRANSCRANIAL MAGNETIC STIMULATION (TMS) DEVICE

(71) Applicants: Amir Katz, Haifa (IL); Eyal Baror, Shoham (IL)

(72) Inventors: Amir Katz, Haifa (IL); Eyal Baror, Shoham (IL)

(73) Assignee: NEURONIX LTD., Yokneam Illit (IL)

(21) Appl. No.: 13/882,671

(22) PCT Filed: Nov. 1, 2011
(86) PCT No.: PCT/IL11/00845

§ 371 Date: Dec. 4, 2013

Related U.S. Application Data
(60) Provisional application No. 61/408,935, filed on Nov. 1, 2010.

(51) Int. Cl. A61N 2/00; A61B 19/00

(52) U.S. Cl. CPC A61N 2/006; A61B 19/54; A61B 19/5212; A61B 19/5202
USPC 600/15

(57) ABSTRACT

The invention provides a system for positioning a TMS stimulating device on a predetermined treatment point on an individual's head. The system includes a cap adapted to be worn on the individual's head. The system also includes one or more markers adapted to be affixed onto an outer surface of the cap that are used in positioning a TMS stimulating device on or adjacent to the treatment point. The markers may be provided with a device that generates a detectable signal. The system could then include a detector associated with the TMS stimulating device that detects the signals generated by the markers. Signals detected by the detector are analyzed in order to determine a position of the TMS stimulating device relative to the treatment point.

### FIELD OF THE INVENTION

The present invention relates to medical devices, and more specifically to such devices for the treatment of neurological conditions.

### BACKGROUND OF THE INVENTION

Neurological and psychiatric conditions are usually managed by drugs and medications. However, over the past decades, it has been found that electric or magnetic stimulation may also be a powerful tool in the treatment of several of such conditions. For example, it was found that Electro-Convulsive Therapy (ECT) is a highly effective tool in the treatment of drug-resistant depression. While the mechanism of action is not entirely clear, it has been speculated to include induction of electric currents that induce simultaneous depolarization of neurons. However, ECT requires general anesthesia, and entails significant side-effects such as memory loss, and others.

It was later found that other, less-aggressive, methods can sometimes be used. One such technique is TMS (Transcranial Magnetic Stimulation), which involves rapidly varying a magnetic field adjacent to the head by means of a special coil. Since a time-varying magnetic field induces an electric current as well as an electric potential, a similar effect to that of ECT is generated inside the brain. The TMS effect can often be achieved without some of the side-effects of ECT.

In addition to depression, it has also been found that TMS may be effective in the treatment of other conditions, such as depression, schizophrenia, bi-polar disorder and Alzheimer type dementia. In order to achieve a clinical effect, specific regions have to be stimulated. For example, for depression, the treatment typically includes depolarization of the Right Dorso-Lateral Pre-Frontal region. For Alzheimer's disease, the treatment typically includes depolarization of the right/left dorso-lateral pre-frontal regions, right/left somatomotory parietal regions, broca, and the Wernicki.

The process of TMS typically involves several steps. First, a scan of the brain is obtained, and the points of interest in the brain to be stimulated are determined. Several types of brain scans are known that may be used in TMS, such as MRI, f-MRI, perfusion-MRI, diffusion-MRI, X-radiation, CT, Ultrasound, and EEG. The region in the brain is to be treated is indicated on the brain scan. The identification can be performed manually or automatically by appropriate software. A point may then identified in the brain region to be treated, which is referred to as the “target point” for the TMS. Finally, a treatment point on the head is determined which is the location where the stimulatory TMS coil is to be positioned in order to deliver the stimulation to brain region to be treated. The TMS coil is then positioned on the treatment point.

The magnetic stimulation used in TMS is weaker than the direct electric current stimulation used in ECT, so that the brain volume affected by the magnetic stimulation (referred to herein as “the focal volume”) is typically smaller than the brain volume affected in ECT. In the case of a TMS coil, the focal volume is typically a cube having an edge between 1 to 2 cm. Outside of this limited focal volume, the magnetic field is not strong enough to cause depolarization of neurons, and hence no clinical effect is achieved outside the focal volume. Thus, once the focal volume in the brain to be treated has been determined, the TMS coil has to be positioned at an appropriate location on the head, referred to here as the “treatment point”, so that a magnetic field of sufficient intensity can be created in the focal volume. Since the focal volume in TMS is typically small than the brain volume affected in ECT, positioning of the coil in TMS usually requires more precision than is required in the placement of ECT electrodes. However, positioning of the TMS device in the vicinity of the treatment point typically obscures the treatment point, thus making it difficult to ascertain whether or not the TMS treatment device is properly positioned on the treatment point.

One method for positioning a TMS stimulating device uses a stereo pair of cameras that is positioned in front of the patient's head. As the TMS simulating device is moved towards the treatment point, a processor analyzes images obtained by the cameras to continuously monitor the relative position of the TMS stimulating device, the patients head and the treatment point. The processor informs the user when the TMS stimulating device is appropriately positioned at the treatment point.

### SUMMARY OF THE INVENTION

The present invention provides a system for positioning a TMS stimulating device on a predetermined treatment point on an individual's head. The system of the invention comprises a cap adapted to be worn on the individual's head and one or more markers adapted to be affixed onto the outer surface of the cap. The cap is donned by the individual being treated. The treatment point on the head where the TMS stimulation device is to be positioned may be marked on the cap. One or more of the markers are attached to the cap at one or more predetermined locations. The markers are positioned on the cap in a predetermined relationship to the treatment point. Then, the TMS stimulation device is positioned on the cap at a predetermined orientation relative to the markers. The TMS treatment can then commence.

In one embodiment of the invention, the markers are placed on the cap in locations that are not obscured by the TMS stimulation device. The markers thus facilitate correct postponing of the TMS stimulation device when the treatment point is obscured by the TMS stimulation device. In another embodiment, the markers generate signals that are detected by a detector associated with the TMS stimulation device. Signals detected by the detector are analyzed by a processor to determine the position of the TMS device relative to the markers, which may be obscured by the TMS stimulation device.

The invention also provides a TMS system having a TMS stimulating device and comprising a system of the invention for positioning the TMS stimulating device on a predetermined treatment point on an individual's head.

The invention also provides a method for positioning a TMS stimulation device on an individual's head in a predetermined orientation to a treatment point. In the method of the invention, a cap of the system of the invention is placed on the individual's head. One or more markers are placed onto the outer surface of the cap in a predetermined orientation to the treatment point. The TMS stimulation device is then placed on or adjacent to the individual's head in a predetermined orientation relative to the affixed markers. The TMS treatment can then begin.

Thus, in one of its aspects, the invention provides a system for positioning a TMS stimulating device on a predetermined treatment point on an individual's head comprising:
(a) a cap adapted to be worn on the individual's head and to be affixed to the head; and
(b) one or more markers adapted to be affixed onto an outer surface of the cap.

The markers are adapted to be affixed onto the outer surface of the cap, for example, by means of an adhesive layer or by means of sewing.

In one embodiment, each of the markers is provided with a device that generates a detectable signal. Each of the markers may generate a different signal. The system may then include a detector configured to detect the signals generated by each of the markers. The detector may be adapted to be attached to a TMS stimulation device, or may be integral with the TMS stimulation device. The detector may generate one or more signals indicative of signals detected by the detector from one or more of the markers. The system may further comprise a processor configured to analyze the signals generated by the detector and to determine a position of a TMS stimulation device associated with the probe relative to the markers and the treatment point. The system may further comprise a CRT screen and the processor may be further configured to display the position of the TMS stimulation device associated with the detector relative to the treatment point on the CRT screen. The processor may also be configured to generate a sensible signal when the TMS stimulation device is in a predetermined position relative to the treatment point.

The probe may include, for example, a light source and a camera that detects light from the light source reflected from the signal generator of each of the deployed markers. Each of the markers may reflect light of a different wavelength. ADditionally or alternatively, The signal generators may be RF transponders, in which case, the probe may generate an RF signal that that induces each of the RF transponders to generate an individual RF signal that is detected by the probe. As yet other alternatives, the signal generators may generate a signal selected from an electromagnetic signal, a magnetic field, and an electric field.

The invention also provides a TMS stimulation system having a TMS stimulating device and comprising a system of the invention for positioning the TMS stimulating device on a predetermined treatment point on an individual's head.

The invention further provides a method for positioning a TMS stimulating device on a predetermined treatment point on an individual's head comprising:
(a) placing a cap on the individual's head;
(b) affixing one or more markers onto an outer surface of the cap in a predetermined orientation to the treatment point; and
(c) positioning the TMS stimulation device on or adjacent to the individual's head in a predetermined orientation relative to the affixed markers.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows a system for positioning a TMS stimulation device on an individual's head, in accordance with embodiment of the invention;

FIG. 2a shows the cap of the system of FIG. 1 after worn by an individual to be treated;

FIG. 2b shows the cap of the system of FIG. 1 being worn be the individual to be treated after affixing markers to the cap;

FIG. 2c shows the cap of the system of FIG. 1 being worn by the individual to be treated after postponing the TMS stimulation device;

FIG. 3 shows a system for positioning a TMS stimulation device on an individual's head, in accordance with embodiment of the invention;

FIG. 4a shows the cap of the system of FIG. 3 after worn by an individual to be treated;

FIG. 4b shows the cap of the system of FIG. 3 being worn be the individual to be treated after affixing markers to the cap; and

FIG. 4c shows the cap of the system of FIG. 3 being worn by the individual to be treated after postponing the TMS stimulation device;

### DETAILED DESCRIPTION OF EMBODIMENTS

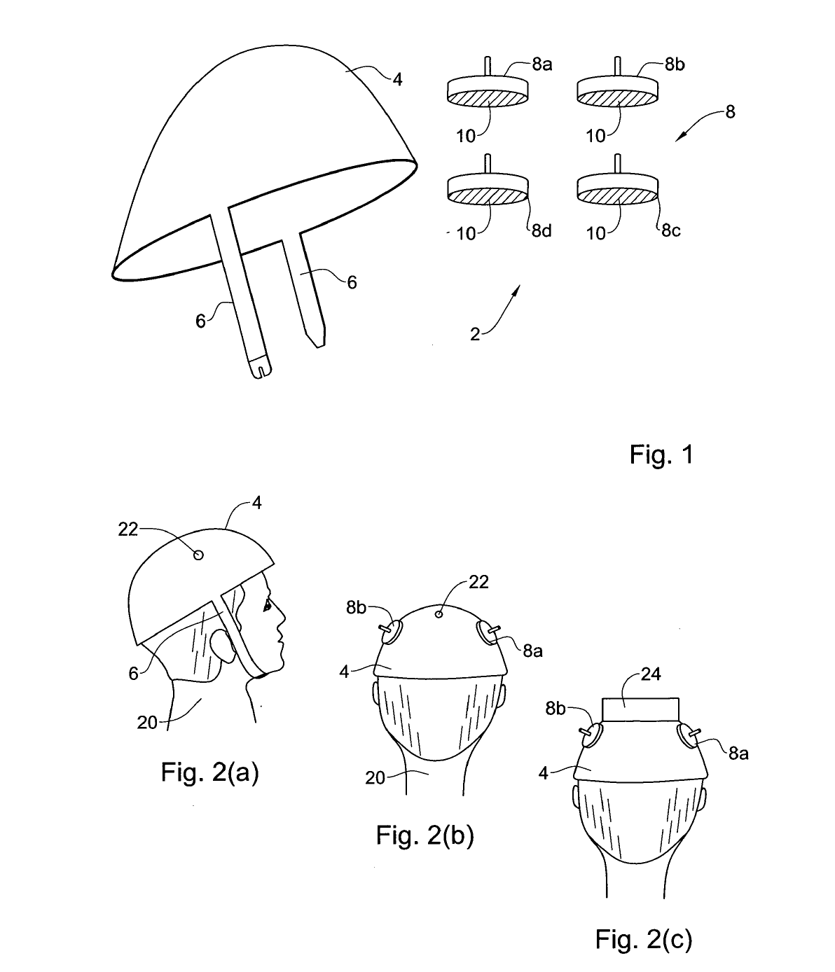

FIG. 1 shows a system 2 for positioning a TMS stimulating device on a predetermined treatment point on an individual's head in accordance with one embodiment of the invention. The system 2 comprises a cap 4 adapted to be worn on the individual's head, and to be rigidly affixed to the head, for example, by means of a chin strap 6. The cap 4 may be made from a thin layer of a flexible material such as rubber or fabric.

The system 2 further comprises one or more markers 8. Four markers, 8a, 8b, 8c, and 8d are shown in FIG. 1. This is by way of example only, and the system may comprise any number of markers as required in any application. Each of the markers 8 is adapted to be affixed onto the outer surface of the cap 4. For example, a bottom surface 10 of each marker may be provided with an adhesive layer that allows the marker to adhere to the cap 4. Alternatively, the markers may be adapted for being sewn to the cap or may be integral with the cap.

As shown in FIG. 2a, in use, the cap 4 is donned by an individual 20 to be treated and the chin strap 6 buckled to prevent movement of the cap 4 on the head. The treatment point 22 on the head where the TMS stimulation device is to be positioned may be marked on the cap 6, for example, using an appropriate marking pen. As mentioned above, when the TMS stimulation device is moved towards the treatment point 22, the treatment point 22 becomes obscured by the TMS stimulation device. One or more of the markers 8 are attached to the cap 4 at one or more predetermined locations that are not obscured by the TMS stimulation device. FIG. 2b shows the individual 20 wearing the cap 4 after deployment of two of the markers 8a and 8b. This is by way of example only, and any number of the markers 8 may be deployed on the cap 6 as required in any application. The markers 8a and 8b are positioned on the cap in a predetermined relationship to the treatment point 22. For example, the markers 8a and 8b may be place on the cap 4 so that the markers 8a and 8b and the treatment point 22 lie in the same vertical plane with the distance from the treatment point to each of the markers being the same for each marker. Then, as shown in FIG. 2c, a TMS stimulation device 24 is positioned on the cap 4 at a predetermined orientation relative to the markers 8a and 8b, which are not obscured by the TMS stimulation device 24. The TMS stimulation device may be moved manually or by a robotic device. The TMS treatment can then commence.

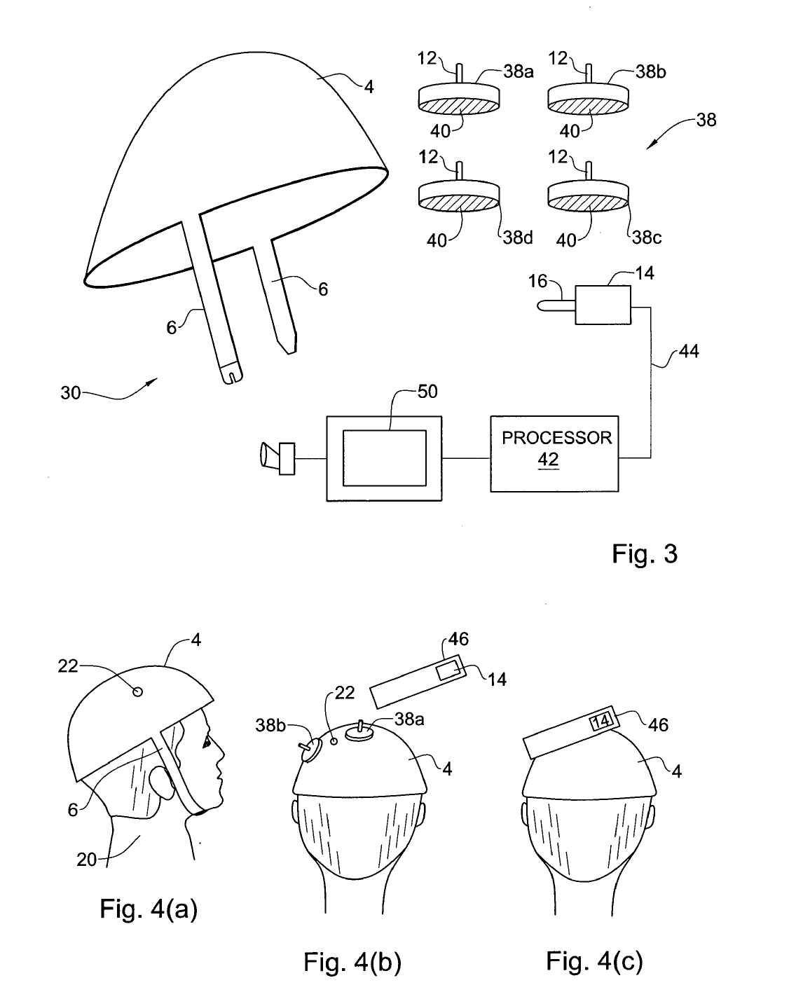

FIG. 3 shows a system 30 for positioning a TMS stimulating device on a predetermined treatment point on an individual's head, in accordance with another embodiment of the invention. Like the system 2 shown in FIGS. 1 and 2, the system 30 also comprises a cap 4 adapted to be worn on the individual's head, and to be rigidly affixed to the head, for example, by means of a chin strap 6.

The system 3 further comprises one or more markers 38. Four markers, 38a, 38b, 38c, and 38d are shown in FIG. 3. This is by way of example only, and the system may comprise any number of markers as required in any application. Each of the markers 38 is adapted to be affixed onto the outer surface of the cap 4. For example, a bottom surface 40 of each marker may be provided with an adhesive layer that allows the marker to adhere to the cap 4. Each of the markers 38 is also provided with a device 12 that generates a detectable signal. The signals generated by the markers 38 are preferably different for each of the markers 38.

The system 30 also comprises a detector 14 that includes a probe 16 for detecting a signal generated by each of the markers 38. The detector 14 may be adapted to be attached to a TMS stimulation device or may be integral with the TMS stimulation device.

In one embodiment, the probe includes a light source and a camera that detects light from the light source reflected from the signal generator of each of the deployed markers. Each of the deployed markers may have a different color, so that the different deployed markers can be identified in images obtained by the camera. In another embodiment, the signal generators 12 are RF transponders, and the probe 14 generates an RF signal that that induces each of the RF transponders to generate an individual RF signal that is detected by the probe 14. In other embodiments, the signal generators generate an infrared signal an ultraviolet signal, a magnetic field, or an electric field, where the probe 14 is appropriately adapted to detected the generated signals.

As shown in FIG. 4a, in use, the cap 4 is donned by an individual 20 to be treated and the chin strap 6 buckled to prevent movement of the cap 4 on the head. The treatment point 22 on the head where the TMS stimulation device is to be positioned may be marked on the cap 6, for example, using an appropriate marking pen. One or more of the markers 38 are attached to the cap 4 at one or more predetermined locations that may or not be obscured by the TMS stimulation device. FIG. 4b shows the individual 20 wearing the cap 4 after deployment of two of the markers 38a and 38b. This is by way of example only, and any number of the markers 38 may be deployed on the cap 4 as required in any application. The markers 38a and 38b are positioned on the cap in a predetermined relationship to the treatment point 22. For example, the markers 38a and 38b may be place on the cap 4 so that the markers 38a and 38b and the treatment point 22 lie in the same vertical plane with the distance from the treatment point to each of the markers being the same for each marker. A TMS stimulation device 46, together with the detector 14, is then moved towards the treatment point 22. The TMS stimulation 14 device may be moved manually or by a robotic device.

As the TMS stimulator approaches the treatment point 22, the detector 14 detects the signals generated by each of the deployed markers 38. Referring again to FIG. 3, the signal detector 14 generates one or more signals indicative of signals detected by the detector 14. The signals generated by the probe 14 are input to a processor 42 over a transmission line 44, that may be a wired transmission line or a wireless transmission line. The processor 42 is configured to analyze the signals generated by the detector 14 and to determine the position of the stimulation device 46 relative to the deployed markers and hence relative to the treatment point. The position of the TMS stimulation device 46 relative to the treatment point 22 may be presented on a CRT screen 50, and a sensible signal may be produced, for example sounding an alarm 52 when, when the TMS stimulation device 46 is appropriately positioned relative to the treatment point (shown in FIG. 4c). The TMS treatment can then commence.

1. A system for positioning a TMS stimulating device on a predetermined treatment point on an individual's head comprising:
(a) a cap adapted to be worn on the individual's head; and
(b) one or more markers adapted to be affixed onto an outer surface of the cap.

2. The system according to claim 1 wherein the one or more markers are adapted to be affixed onto the outer surface of the cap by an adhesive layer.

3. The system according to claim 2 wherein the one or more markers are adapted to be affixed onto the outer surface of the cap by sewing.

4. The system according to claim 1, wherein each of the one or more markers is provided with a device that generates a detectable signal.

5. The system according to claim 4 wherein each of the one or more markers generates a different signal.

6. The system according to claim 4 further comprising a detector configured to detect the signals generated by each of the one or more markers.

7. The system according to claim 6 wherein the detector is adapted to be attached to a TMS stimulation device.

8. The system according to claim 6 wherein the detector is integral with a TMS stimulation device.

9. The system according to claim 6 wherein the detector generates one or more signals indicative of signals detected by the detector from one or more of the one or more markers.

10. The system according to claim 9 further comprising a processor configured to analyze the signals generated by the detector and to determine a position of a TMS stimulation device associated with the probe relative to the one or more markers and the treatment point.

11. The system according to claim 10 further comprising a CRT screen and wherein the processor is further configured to display the position of the TMS stimulation device associated with the detector relative to the treatment point on the CRT screen.

12. The system according to claim 10 wherein the processor is further configured to generate a sensible signal when the TMS stimulation device is in a predetermined position relative to the treatment point.

13. The system according to claim 6 wherein the probe includes a light source and a camera that detects light from the light source reflected from the signal generator of each of the deployed markers.

14. The system according to claim 13 wherein each of the markers reflects light of a different wavelength.

15. The system according to claim 6 wherein the signal generators are RF transponders, and the probe generates an RF signal that that induces each of the RF transponders to generate an individual RF signal that is detected by the probe.

16. The system according to claim 6 wherein the signal generators generate a signal selected from an electromagnetic signal, a magnetic field, and an electric field.

17. A TMS stimulation system having a TMS stimulating device and comprising a system according to claim 1 for positioning the TMS stimulating device on a predetermined treatment point on an individual's head.

18. A method for positioning a TMS stimulating device on a predetermined treatment point on an individual's head comprising:
(a) placing a cap on the individual's head;
(b) affixing one or more markers onto an outer surface of the cap in a predetermined orientation to the treatment point; and
(c) positioning the TMS stimulation device on or adjacent to the individual's head in a predetermined orientation relative to the affixed markers.
